(19) United States
(12) Patent Application Publication
(10) Pub. No.: US 2026/0165935 A1
(43) Pub. Date: Jun. 18, 2026

(54) AI-Driven Oral Photoprotective Compositions, Systems, And Methods Of Designing, Synthesizing, And Detecting The Same

(71) Applicant: Scott White, Minneapolis, MN (US)

(72) Inventor: Scott White, Minneapolis, MN (US)

(21) Appl. No.: 19/241,291

(22) Filed: Jun. 17, 2025

Related U.S. Application Data
(63) Continuation-in-part of application No. 19/220,706, filed on May 28, 2025.

(51) Int. Cl. A61K 8/49; A61Q 17/04; B01D 15/08; G01N 30/02; G01N 30/72; G16C 20/50; G16C 20/70; H01J 49/00

(52) U.S. Cl. CPC A61K 8/4926; A61Q 17/04; B01D 15/08; G01N 30/7233; G16C 20/50; G16C 20/70; H01J 49/004; A61K 2800/92; G01N 2030/027

(57) ABSTRACT

Oral compositions and methods for providing topical photoprotection against ultraviolet (UV) radiation are disclosed, wherein active photoprotective agents are designed, optimized, synthesized, and detected using artificial intelligence (AI) driven platforms. The invention encompasses AI-driven methods for designing novel photoprotective molecules (e.g., “Compound X-Sweat”) with specific physicochemical properties conducive to glandular excretion (via sweat and/or sebum) and optimized UV absorption characteristics. The invention further includes the novel AI-designed compounds themselves, plausible methods for their chemical synthesis, and synergistic photoprotective systems designed by AI for broad-spectrum coverage, multi-pathway excretion, and/or enhanced glandular excretion of photoprotective agents, with synergy being quantitatively defined. Additionally, methods for detecting and quantifying these agents on a subject's skin surface following oral administration are provided, serving as a companion diagnostic and means for verifying efficacy and infringement. The AI-designed agent(s), following oral administration, are excreted onto the skin surface, forming a protective layer or achieving a localized concentration effective to absorb or scatter UV radiation or provide antioxidant effects, thereby enabling a systemic route to persistent, broad-spectrum, and user-friendly photoprotection without the drawbacks of topical sunscreen reapplication.

### CROSS-REFERENCE TO RELATED APPLICATIONS

This application is a Continuation-in-Part of U.S. Non-Provisional patent application Ser. No. 19/220,706, filed on May 28, 2025, which in turn claims the benefit of U.S. Provisional Application No. 63/805,464, filed on May 14, 2025. The entire contents of both of the aforementioned applications are hereby incorporated by reference herein.

### BACKGROUND OF THE INVENTION

### 1. Field of the Invention

The present invention relates generally to the fields of photoprotection, dermatology, and computational chemistry. More specifically, it pertains to orally administered compositions that provide protection against the deleterious effects of ultraviolet (UV) radiation on mammalian skin by delivering active photoprotective agents to the external surface of the skin via physiological excretion pathways, such as sweat and/or sebum. The invention is particularly directed to artificial intelligence (AI) driven methods for designing, optimizing, synthesizing, and detecting said agents and compositions, representing a significant advancement in creating tailored photoprotective solutions.

### 2. Description of Related Art

Exposure to solar UV radiation is a primary cause of various skin damage modalities, including erythema (sunburn), premature aging (photoaging), and the development of skin cancers. Conventional methods of photoprotection, predominantly topical sunscreens, suffer from several well-documented limitations. These include the inconvenience of application and reapplication, incomplete coverage, removal by sweating or abrasion, unpleasant cosmetic feel (e.g., greasiness, white residue), and potential for local irritation. Furthermore, there are growing environmental concerns regarding the impact of certain chemical UV filters washed from the skin into aquatic ecosystems.

While some oral photoprotective agents exist, their mechanisms primarily involve systemic antioxidant effects, DNA repair enhancement, or anti-inflammatory actions within the deeper layers of the skin or systemically. These agents typically do not provide a direct physical or chemical barrier on the skin surface by absorbing or scattering UV radiation in the same manner as topical sunscreens. Consequently, there remains a significant unmet need for improved, convenient, and efficacious methods of photoprotection that address the limitations of current approaches.

Furthermore, while certain endogenous molecules with UV-absorbing properties, such as kynurenic acid, have been identified in human sweat and are known to be part of the skin's natural UV response pathways, their potential has not been heretofore harnessed or optimized through a dedicated oral formulation designed to achieve a therapeutically significant photoprotective layer on the skin surface.

The parent application, U.S. Non-Provisional application Ser. No. 19/220,706, addressed this need by disclosing the foundational concept of an “inside-out” delivery mechanism: an ingestible composition that delivers photoprotective agents to the skin surface via sweat and/or sebum. The parent application identified candidate compounds (e.g., gadusol, mycosporine-like amino acids) and the general physicochemical properties conducive to this excretion pathway.

The present invention builds substantially upon this foundation by introducing and detailing novel inventions centered on the use of Artificial Intelligence to design superior molecules and systems with optimized properties for glandular excretion and photoprotection. Furthermore, this invention provides methods for their chemical synthesis, which is crucial for enabling claims to novel chemical entities, and methods for their detection and quantification on the skin surface. These additions thereby enable a complete and robust technological platform and overcome potential deficiencies in claiming novel compounds without an enabling disclosure of their synthesis, a critical aspect for securing strong intellectual property rights. The strategic introduction of AI-driven design, coupled with synthetic enablement and detection methodologies, represents a transformative step beyond the concepts disclosed in the parent application, offering pathways to previously unattainable levels of precision and efficacy in oral photoprotection.

### SUMMARY OF THE INVENTION

The present invention provides a significant advancement over the prior art, including the parent application, by detailing AI-driven platforms and methods for creating, synthesizing, validating, and utilizing novel oral photoprotective solutions. The invention encompasses several distinct but interrelated inventive aspects, each capable of forming the basis of a divisional application, thereby providing a strategic framework for comprehensive intellectual property protection. This multifaceted approach is intended to establish a robust “picket fence” of patents around the core technology, maximizing its defensive and offensive value.

In one aspect, the invention provides a method for designing or identifying a candidate photoprotective molecule optimized for glandular excretion (e.g., via sweat or sebum) and UV protection by using one or more trained artificial intelligence (AI) models operating on a computing system. This method involves defining target property profiles, including specific physicochemical characteristics (e.g., molecular weight, lipophilicity (LogP), water solubility, topological polar surface area (TPSA)) conducive to the desired excretion pathway, and desired UV absorption spectra (e.g., target λmax range, target molar extinction coefficient (ε)). Generative AI models (e.g., Generative Adversarial Networks (GANs), Variational Autoencoders (VAEs), Transformer models) are employed to create novel chemical structures, which are then screened and selected using predictive AI models that assess their conformity to the defined profiles.

In another aspect, the invention provides a novel photoprotective compound, exemplified by, but not limited to, “Compound X-Sweat,” designed by such an AI-driven method. Said compound possesses a unique chemical structure and a profile of physicochemical and photoprotective properties computationally predicted and specifically engineered to be optimized for oral administration, subsequent glandular excretion (e.g., via sweat), and effective UV protection on the skin surface. These AI-designed compounds represent a new class of photoprotective agents tailored from first principles for this specific mode of delivery and action.

In yet another aspect, the invention provides for the novel use of a known compound, Kynurenic Acid, for providing photoprotection. This aspect includes a method of providing topical photoprotection to the skin of a subject by orally administering a composition comprising a therapeutically effective amount of Kynurenic Acid, or a pharmaceutically acceptable salt thereof. Kynurenic Acid, an endogenous human metabolite, serves as a real-world example of a compound that functions via the glandular excretion mechanism central to the present invention, and its use as a formulated oral photoprotective agent represents a novel application of this known molecule.

In a further aspect, the invention provides a method for designing a synergistic photoprotective system using AI-driven multi-objective optimization. This method selects or designs combinations of agents, which may include AI-designed compounds or known agents, to achieve complementary benefits. Such benefits include, but are not limited to, broad-spectrum UV coverage, enhanced photostability, dual-pathway excretion (e.g., one agent optimized for sweat excretion, another for sebum excretion), and/or synergistic enhancement of glandular excretion of one photoprotective agent by another co-administered agent. A key feature of this aspect is the quantitative definition of synergy. For example, synergistic UV spectral coverage is defined as the combination achieving a mean UV absorbance across a specified wavelength range (e.g., 290-380 nm) that is at least 15% greater than the calculated additive absorbance of the individual components at their predicted skin surface concentrations. Similarly, synergistic excretion enhancement may be defined as a co-administered agent increasing the measured skin surface concentration of a primary photoprotective agent by at least a predetermined percentage (e.g., 20%) compared to the administration of the primary agent alone. This quantitative definition transforms a previously vague concept into a concrete, defensible claim limitation.

The invention also provides the synergistic photoprotective compositions identified or designed by this AI method, comprising at least two photoprotective agents exhibiting such quantitatively defined synergy (e.g., in UV coverage or excretion enhancement). These compositions offer enhanced performance profiles unattainable by single agents or simple additive mixtures.

In yet another aspect, the invention provides a method for synthesizing a novel AI-designed photoprotective compound, exemplified by a plausible chemical synthesis route for “Compound X-Sweat.” This discloses a plausible synthetic pathway sufficient to enable one of skill in the art to the physical embodiment of the computationally designed molecule, satisfying the critical requirements for written description and enablement for composition of matter claims. The inclusion of such synthetic methods directly addresses a common point of rejection for patents claiming novel compounds based solely on computational design.

In still another aspect, the invention provides a method for providing rapid photoprotection comprising administering a single oral loading dose of a photoprotective agent as described herein, wherein the single loading dose is sufficient to cause the excretion of the agent onto the skin surface at a therapeutically effective concentration within 2 to 6 hours of administration.

In a final substantive aspect, the invention provides a method for detecting and quantifying the concentration of an orally administered photoprotective agent (such as “Compound X-Sweat”) on a subject's skin surface following oral administration. This method, which may be embodied as a companion diagnostic, serves multiple strategic purposes: it allows for the validation of efficacy, confirms the intended mechanism of action (i.e., glandular excretion and deposition onto the skin surface), and provides a clear, practical means for demonstrating infringement of method-of-use claims. This aspect adds significant value by linking the therapeutic effect to a measurable outcome and providing tools for enforcement.

Further aspects of the invention include methods of providing photoprotection to a subject by orally administering a therapeutically effective amount of the AI-designed compounds or synergistic compositions described herein. These methods aim to achieve a measurable increase in the Minimal Erythema Dose (MED) or other quantifiable metrics of photoprotection.

### BRIEF DESCRIPTION OF THE SEVERAL VIEWS OF THE DRAWING(S)

FIG. 1 is a conceptual block diagram illustrating the overall process of the invention, from oral ingestion of the composition to the interaction of excreted photoprotective agents with UV radiation on the skin surface (based on parent application U.S. Non-Provisional application Ser. No. 19/220,706).

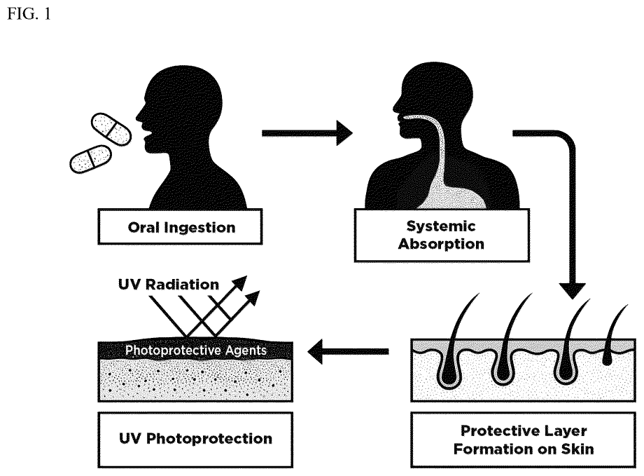

FIG. 2 shows the chemical structures of exemplary known photoprotective agents discussed in the parent application (U.S. Non-Provisional application Ser. No. 19/220,706), which may serve as comparators or components in AI-designed synergistic systems of the present invention.

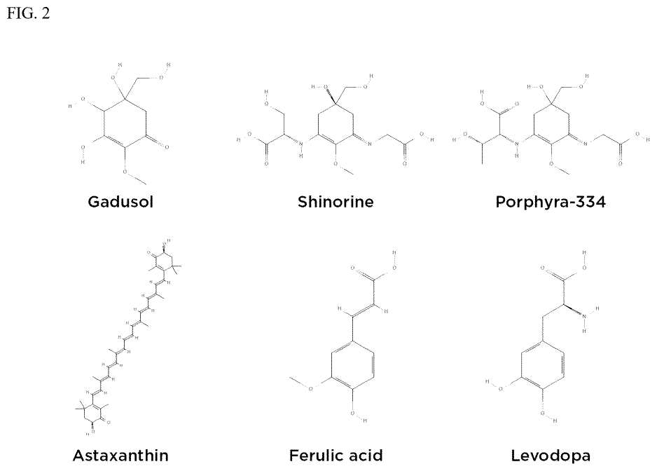

FIG. 3 is a prophetic graph illustrating the predicted concentration of a photoprotective agent on the skin surface over time following oral administration (based on parent application U.S. Non-Provisional application Ser. No. 19/220,706).

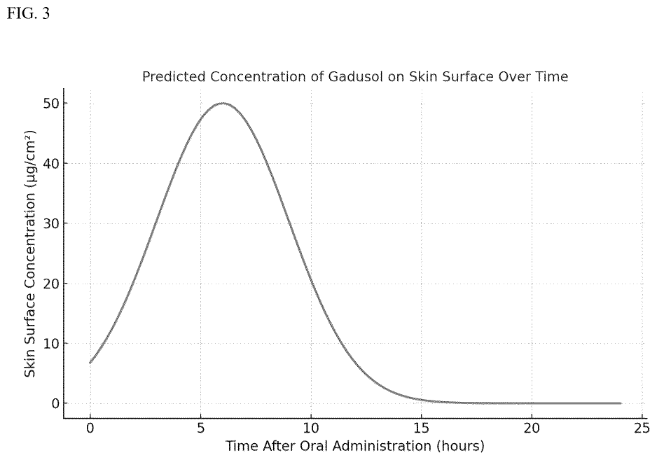

FIG. 4 is a prophetic graph illustrating the UV absorption spectrum of excreted photoprotective agents on a skin-mimicking substrate. The AI-designed synergistic systems of the present invention aim to produce optimized spectra with broader and more uniform UV coverage compared to individual agents or non-optimized combinations (expanded from parent application U.S. Non-Provisional application Ser. No. 19/220,706).

FIG. 5 is a conceptual diagram illustrating the steps of the prophetic companion diagnostic method for quantifying an AI-designed photoprotective agent (e.g., “Compound X-Sweat”) on the skin surface, from skin sampling using techniques like tape stripping, to sample processing, analytical quantification (e.g., via LC-MS/MS), and calculation of surface concentration. This figure is specific to the new matter introduced in this Continuation-in-Part application and provides crucial visual support for the detection and quantification method.

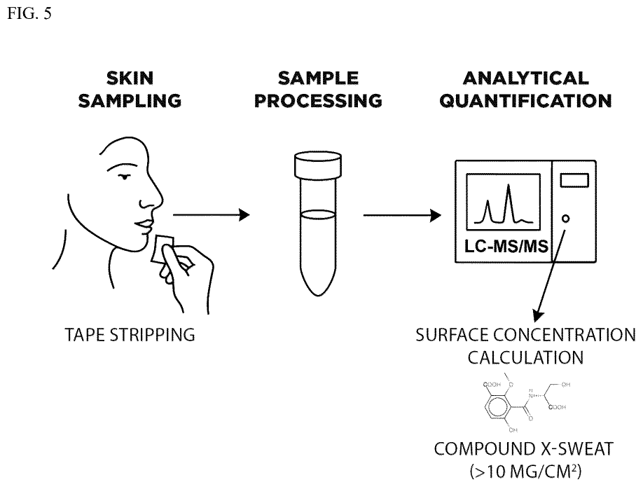

FIG. 6 is a block flowchart illustrating the key steps of the AI-driven method for designing a novel photoprotective molecule, in accordance with one aspect of the present invention. The process begins with defining target property profiles (e.g., for glandular excretion and UV absorption), followed by employing generative AI models to create novel chemical structures. These structures are then computationally screened and ranked by predictive AI models, leading to the selection of a lead candidate molecule (e.g., “Compound X-Sweat”) for synthesis and experimental validation. This figure provides essential support for the novel AI design method claims and is specific to the new matter introduced in this Continuation-in-Part application.

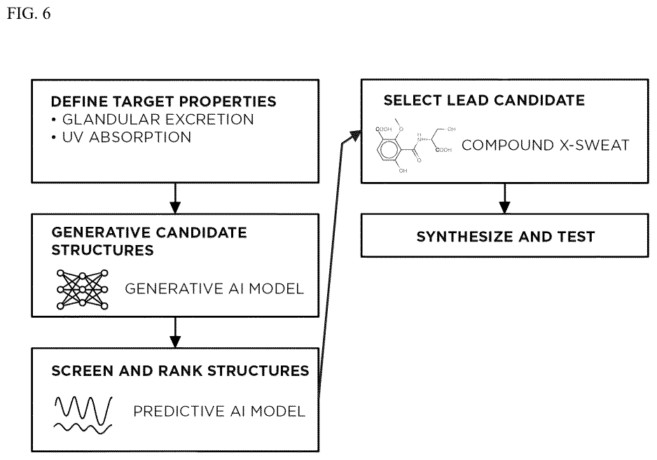

### DETAILED DESCRIPTION OF THE INVENTION

The present invention is directed to AI-driven oral photoprotective compositions and systems, and methods of designing, synthesizing, detecting, and using the same. The core innovation lies in the strategic application of Artificial Intelligence to design and optimize photoprotective agents and systems that are administered orally and subsequently excreted onto the skin surface via sweat and/or sebum to provide topical photoprotection, building upon foundational concepts established in the parent application.

### 1. Foundational Concepts: Systemic Administration for Topical Photoprotection Via Glandular Excretion

The cornerstone of the present invention, as introduced in the parent application U.S. Non-Provisional application Ser. No. 19/220,706 (incorporated herein by reference), is a novel method for delivering photoprotective compounds to the external surface of the skin. Unlike traditional topical sunscreens applied externally, or conventional oral photoprotectants that primarily exert systemic antioxidant or DNA repair effects within the body or deeper skin layers, the present invention utilizes the body's natural excretory pathways—specifically eccrine sweat glands and sebaceous glands—to transport orally administered active agents to the skin surface. Once on the skin surface, these agents can form a protective film, absorb UV radiation, scatter UV radiation, or provide potent localized antioxidant defense, thereby protecting the underlying skin cells from UV-induced damage.

It is established that various endogenous and exogenous substances, including drugs and their metabolites, can be excreted from the body via sweat and sebum following systemic administration. The mechanisms by which compounds are excreted into sweat and sebum are complex and can involve passive diffusion from the blood into the gland and/or active transport processes mediated by transporters such as those from the ATP-Binding Cassette (ABC) and Solute Carrier (SLC) families present in glandular cells. The efficiency of this process for a given compound is influenced by its physicochemical properties, including molecular weight (MW), lipophilicity (LogP or LogD), water solubility, pKa, and charge at physiological pH. Generally, smaller, more water-soluble compounds are favored for sweat excretion, while more lipophilic compounds tend to partition more readily into sebum. The parent application provides further details on these mechanisms and properties.

### 2. The AI-Driven Design, Optimization, Synthesis, and Detection Platform

Building upon the core innovation of glandular excretion for topical delivery, the present invention introduces an AI-centric platform for the design, optimization, synthesis, and detection of superior photoprotective agents and systems. This platform represents a significant leap forward, enabling the creation of molecules and formulations specifically engineered for this unique application.

### A. AI Framework and Methodologies for Molecule and System Design

An overarching AI pipeline is employed, integrating various computational models and platforms to achieve the design and optimization goals. This pipeline typically involves the following components:

In one component, the pipeline utilizes data inputs. These inputs can include chemical structure information (e.g., SMILES strings) for known compounds or human-designed candidates, human-defined Target Product Profiles (TPPs) specifying desired physicochemical properties, UV absorption characteristics, ADMET (Absorption, Distribution, Metabolism, Excretion, Toxicity) profiles, and potentially experimental data from related compounds or in vitro/ex vivo studies.

In another component, various categories of AI models are utilized. These can include, but are not limited to, generative models and predictive models.

One category of AI models includes generative models, which are employed for the de novo design of novel molecular structures (e.g., “Compound X”). Examples of suitable generative models include Generative Adversarial Networks (GANs), Variational Autoencoders (VAEs), Recurrent Neural Networks (RNNs), Transformer-based architectures, and Large Language Models (LLMs) adapted for molecular design (e.g., MOLLM). These models learn from existing chemical data to propose new molecules with desired characteristics.

Another category of AI models includes a suite of predictive models, which are used for rapidly assessing key properties of the generated or existing candidate structures. These predictive models can be configured to assess a variety of characteristics, as detailed in the following paragraphs.

In one embodiment, the predictive models are used to assess physicochemical properties. Such properties may include, but are not limited to, molecular weight (MW), lipophilicity (LogP), water solubility, topological polar surface area (TPSA), hydrogen bond donor (HBD)/acceptor (HBA) counts, and the acid dissociation constant (pKa). Example tools and platforms for such predictions include SwissADME, pkCSM, ADMET-AI, and RDKit.

In another embodiment, the predictive models are used to assess ADMET characteristics (Absorption, Distribution, Metabolism, Excretion, Toxicity). These may include oral bioavailability, plasma protein binding, cytochrome P450 (CYP) interactions, clearance pathways, and potential toxicities such as hERG inhibition or Ames mutagenicity. Tools like ADMETLab 3.0 may be used for broader ADMET profiling.

In yet another embodiment, the predictive models are used to assess UV absorption spectra. This includes predicting the maximum absorption wavelength (λmax) and the molar extinction coefficient (ϵ). Example tools for these predictions include ML_UVvisModels and quantum mechanical methods like Density Functional Theory (DFT) or Time-Dependent DFT (TD-DFT).

In a further embodiment, the predictive models are used to assess interactions with biological transporters or skin permeability factors. These predictions can be used to identify candidates with enhanced potential for glandular excretion, for example by predicting favorable interactions with transporters known to be present in glandular cells (e.g., ABC transporters).

In some embodiments, the AI framework may further utilize Multi-Objective Optimization (MOO) algorithms. These algorithms are crucial for designing synergistic systems or single molecules that must satisfy multiple desired properties simultaneously. MOO algorithms can be employed to optimize for several parameters in parallel, such as dual-pathway excretion (e.g., one agent optimized for sweat and another for sebum), complementary UV absorption spectra, favorable ADMET profiles, and/or the potential for excretion enhancement of one agent by another.

The framework may also incorporate AI-assisted Physiologically Based Pharmacokinetic (PBPK) models. These models are used to predict the rate and extent of glandular excretion and the resulting concentration of an agent on the skin surface following oral administration. Inputs for these models may include AI-predicted compound properties as well as physiological parameters of the subject. Example platforms for implementing such models include PK-Sim/MoBi or custom models developed in programming languages such as R.

The AI pipeline typically involves several processing steps. These steps generally include iterative cycles of: (a) generation or selection of candidate molecules; (b) prediction of their physicochemical, ADMET, and photoprotective properties; (c) evaluation of the predicted properties against target criteria defined in the TPPs; and (d) optimization based on the results of the evaluation.

The process ultimately yields one or more outputs. These outputs may include one or more lead candidate molecules (e.g., “Compound X-Sweat”) or synergistic systems of multiple molecules. The output for each candidate or system typically includes their AI-predicted structures and a comprehensive profile of their relevant predicted properties.

The integration of these diverse AI tools into a rational framework allows for the efficient exploration of vast chemical spaces and the identification or design of photoprotective agents that are specifically optimized for glandular excretion and potent UV protection, a task that would be exceedingly difficult or impossible through traditional discovery methods alone.

TABLE 1
Overview of AI Models, Tools, and Methodologies. The following table provides
a structured overview of the AI toolkit employed in the present invention:

| AI Application | AI Model/Tool | | | Purpose in CIP | |
|---|---|---|---|---|---|
| Area | Type | Key Inputs | Key Outputs | Context | Example Tools |
| Physicochemical | Predictive | SMILES, | MW, LogP, | Initial screening, | SwissADME, |
| Property | Machine Learning | Chemical | Solubility, TPSA, | input for PBPK & | pkCSM, |
| Prediction | (ML) | Structure | HBD/HBA, pKa | generative models | ADMET-AI, |
| | | | | | RDKit |
| ADMET | Predictive ML | SMILES, | Oral | De-risking | SwissADME, |
| Screening | | Chemical | bioavailability, | candidates, input | pkCSM, |
| | | Structure | CYP interactions, | for generative | ADMET-AI, |
| | | | toxicity (Ames, | models | ADMETlab 3.0 |
| | | | hERG) | | |
| UV Spectra | QM methods | SMILES, | λmax, Molar | Assessing | ML_UVvisModels, |
| Prediction | (DFT, TD-DFT), | Chemical | Extinction | photoprotective | GAMESS/ORCA |
| | Predictive ML | Structure | Coefficient (ε) | efficacy, input for | |
| | | | | generative models | |
| De Novo | Generative AI | Target Property | Novel | Designing novel | REINVENT, |
| Molecular Design | (GANs, VAEs, | Profiles | SMILES/Structures | photoprotective | libINVENT, moo- |
| (“Compound X”) | RNNs, LLMs), | (Physicochem, | with predicted | molecules | denovo, |
| | MOO | UV, ADMET) | properties | optimized for | DeepChem |
| | | | | glandular | |
| | | | | excretion | |
| Synergistic | MOO, Predictive | Properties of | Optimized | Designing | Custom |
| System Design | ML for Synergy | individual | combinations/ | systems with | algorithms, |
| | | compounds, | multi-property | enhanced/ | adapting synergy |
| | | interaction data | molecules, | complementary | prediction models |
| | | (e.g., transporter | predicted synergy | protection & | |
| | | modulation, | scores (UV | excretion, or | |
| | | permeability | coverage, | excretion | |
| | | enhancement) | excretion | enhancement | |
| | | | enhancement) | | |
| Glandular | PBPK models | AI-predicted | Predicted skin | In silico validation | PK-Sim/MoBi, R- |
| Excretion | | compound | surface | of excretion | based models |
| Prediction | | properties, | concentration, | mechanism, dose | |
| (PBPK) | | physiological | excretion pathway | prediction | |
| | | parameters | likelihood | | |
| Novel Glandular | Custom AI model | Molecular | Predicted | Addressing | (To be specified if |
| Excretion | (e.g., specialized | descriptors, | sweat/sebum | limitations of | novel |
| Prediction/ | neural network) | potentially | excretion | standard ADMET | methodology is |
| Enhancement | | QSAR-based, | efficiency/ratio, | tools for specific | developed) |
| Prediction | | transporter | predicted | glandular | |
| | | interaction data | excretion | excretion | |
| | | | enhancement | pathways or | |
| | | | factor | predicting | |
| | | | | excretion synergy | |

### B. AT-Optimized Target Product Profiles (TPPs) for Excretion and Photoprotection

The AI-driven design process is guided by specific, human-defined Target Product Profiles (TPPs). These TPPs quantitatively define the desired characteristics of the photoprotective agents or systems to be designed. The following are exemplary, non-limiting embodiments of such TPPs.

In one embodiment, an AI-Optimized Target Profile for Sweat Excretion is defined. The parameters for this profile may include, but are not limited to: a Molecular Weight (MW) in the range of approximately 150 to 350 Da; a lipophilicity (logP) in the range of approximately −2.0 to +0.5; a high water solubility, for example greater than 10 mg/mL; a Topological Polar Surface Area (TPSA) in the range of approximately 60 to 120 Å²; and hydrogen bond donor/acceptor (HBD/HBA) counts of HBD≤4 and HBA≤8.

In another embodiment, an AI-Optimized Target Profile for Sebum Excretion is defined. The parameters for this profile may include, but are not limited to: a Molecular Weight (MW) in the range of approximately 250 to 550 Da; a lipophilicity (logP) in the range of approximately +3.5 to +6.0; a low water solubility, for example less than 0.01 mg/mL, indicating the compound is lipid-soluble; a Topological Polar Surface Area (TPSA) of less than approximately 80 Å²; and hydrogen bond donor/acceptor (HBD/HBA) counts of HBD≤2 and HBA≤4.

In a further embodiment, an AI-Optimized Target Profile for Photoprotection is defined. The parameters for this profile may include one or more of the following: a desired maximum absorption wavelength (λmax) in a specific range, such as 290-320 nm for UVB protection or 320-400 nm for UVA protection, or complementary values for use in synergistic systems; a high molar extinction coefficient (ϵ), for example greater than 15,000 M⁻¹cm⁻¹, and ideally greater than 25,000 M⁻¹cm⁻¹; effective broad-spectrum absorbance across a range such as 290 nm to 380 nm for systems; high photostability; and potentially, relevant antioxidant properties as a co-optimization goal.

In yet another embodiment, an AI-Optimized Target Profile for an “Enhancer Compound E” is defined, wherein the compound is designed for excretion enhancement. The parameters for this profile may include: properties conducive to interaction with glandular transporters (e.g., ABC transporters) or for the modification of skin and/or glandular permeability (e.g., specific terpenes or fatty acids); low intrinsic photoprotective activity, such that the compound acts primarily as an enhancer; and a favorable ADMET profile to ensure it can be safely co-administered with a primary photoprotective agent.

The AI's multi-objective optimization capability is critical for simultaneously achieving these UV targets and the physicochemical properties required for efficient glandular excretion, or for designing an effective excretion enhancer.

TABLE 2
AI-Optimized Target Product Profiles (TPPs). The following table summarizes the target physicochemical
and photoprotective properties used to guide the AI-driven design process:

| | | | Target | |
|---|---|---|---|---|
| | Target for Sweat | Target for Sebum | Photoprotective | Target for Excretion |
| | Excretion (AI- | Excretion (AI- | Profile (AI- | Enhancer Compound |
| Property | Optimized) | Optimized) | Optimized) | (AI-Optimized) |
| Molecular Weight (Da) | e.g., 150-350 Da | e.g., 250-550 Da | N/A (indirectly | Variable, depends on |
| | | | constrained) | mechanism (e.g., <500 |
| | | | | Da) |
| LogP | e.g., −2.0 to +0.5 | e.g., +3.5 to +6.0 | N/A | Variable, depends on |
| | | | | target interaction site |
| Water Solubility | e.g., >10 mg/mL | e.g., <0.01 mg/mL | N/A | Variable |
| TPSA (Å²) | e.g., 60-120 Å² | e.g., <80 Å² | N/A | Variable |
| HBD Count | e.g., ≤4 | e.g., ≤2 | N/A | Variable |
| HBA Count | e.g., ≤8 | e.g., ≤4 | N/A | Variable |
| UV λmax (nm) | N/A | N/A | e.g., 290-320 nm | Preferably minimal UV |
| | | | (UVB), 320-400 nm | absorption |
| | | | (UVA) | |
| Molar Extinction | N/A | N/A | e.g., >20,000, | Preferably low |
| Coefficient | | | ideally >25,000 | |
| (ε, M⁻¹cm⁻¹) | | | M⁻¹cm⁻¹ | |
| Oral Bioavailability | e.g., High (F >60%) | e.g., Moderate to High | N/A | Good to High |
| (Predicted) | | | | |
| Key ADMET Flags | e.g., Low hERG risk, | e.g., Low hERG risk, | N/A | Low toxicity, low |
| (Predicted) | Ames negative | Ames negative | | drug-drug interaction |
| | | | | potential (unless |
| | | | | specific interaction is |
| | | | | desired for |
| | | | | enhancement) |
| Transporter | N/A | N/A | N/A | Predicted activity (e.g., |
| Modulation/ | | | | inhibits efflux pumps |
| Permeability | | | | like P-gp, alters SC |
| Enhancement | | | | lipids) |

### 3. Novel Photoprotective Compounds Designed by Artificial Intelligence (e.g., “Compound X-Sweat”)

A significant aspect of the present invention is the de novo design of novel photoprotective molecules using the AI methodologies described above. These molecules, exemplified herein by “Compound X-Sweat,” are engineered from inception to possess optimal characteristics for potent photoprotective activity and efficient, targeted excretion via specific glandular pathways (e.g., predominantly sweat or predominantly sebum).

“Compound X-Sweat” serves as an illustrative example of such an AI-designed molecule. It is designed to meet a TPP prioritizing sweat excretion and UVB protection. An illustrative chemical structure for “Compound X-Sweat” may be represented by the SMILES string: COc1c(C(═O)N[C@H](CO)C(═O)O)c(O)cc(C(═O)O)c1. The AI-driven design process predicts that this molecule will exhibit properties such as a molecular weight of approximately 250-255 Da, a LogP of approximately −0.5, high water solubility (>10 mg/mL), a UV λmax around 308-310 nm with a molar extinction coefficient (ε) greater than 25,000 M⁻¹ cm⁻¹, and a favorable ADMET profile (e.g., good oral bioavailability, low toxicity risk). AI-assisted PBPK modeling further predicts that such a compound, when administered orally, can achieve therapeutically relevant concentrations on the skin surface (e.g., approximately 15 μg/cm² after 7 days of a 100 mg daily dose) via sweat excretion. Such molecules, designed by AI, represent a departure from repurposing existing compounds and offer the potential for superior performance tailored to the specific application of oral photoprotection via glandular delivery.

### 4. Exemplary Embodiment: Kynurenic Acid as a Glandularly Excreted Photoprotective Agent

In addition to the de novo design of novel molecules, a further aspect of the invention involves the novel use of known compounds that are shown to function via the inventive mechanism. Kynurenic Acid (KYNA) serves as a non-limiting, real-world, and enabling example of such a compound. KYNA is an endogenous metabolite of the amino acid tryptophan and has been identified as a component of the skin's natural defense system.

Crucially, KYNA provides a direct, evidence-based example of the core mechanism of the present invention. Scientific literature has demonstrated that KYNA is endogenously present in human sweat and that its concentration on the skin surface increases significantly in response to physical exercise (see, e.g., Seo et al., Scientific Reports 2017, 7, 13349). This observation confirms that a UV-absorbing molecule can be delivered from within the body to the external surface of the skin via glandular excretion pathways, such as sweat, in physiologically relevant amounts.

Furthermore, KYNA is a known UV chromophore, exhibiting strong absorbance in the UVA range (approximately 320-340 nm) (see, e.g., Plonka et al., J. Photochem. Photobiol. B 2014, 140, 367-371). Its presence on the skin surface following excretion therefore provides a direct, topical photoprotective barrier, consistent with the goals of the present invention.

To further validate the predictive models used in the AI platform of the present invention, Kynurenic Acid was analyzed using a representative in silico tool (SwissADME). The AI-predicted properties for Kynurenic Acid are presented in Table 3 below and compared against the AI-Optimized Target Profile for Sweat Excretion.

TABLE 3
Comparison of AI-Predicted Properties for Kynurenic
Acid against the Target Profile for Sweat Excretion.

| | Target Profile | | Predicted for | | Alignment with |
|---|---|---|---|---|---|
| Property | (Sweat) | | Kynurenic Acid | | Target |
| Molecular Weight | 150-350 | Da | 173.17 | Da | Yes |
| (Da) | | | | | |
| LogP | −2.0 to +0.5 | | 1.31 | | No |
| Water Solubility | High | | Soluble | | Yes |
| TPSA (Å²) | 60-120 | Å² | 50.19 | Å² | No (Slightly |
| | | | | | below) |

| | | | |
|---|---|---|---|
| HBD Count | ≤4 | 1 | Yes |
| HBA Count | ≤8 | 3 | Yes |
| PAINS/Brenk | 0 | 0 | Yes |
| Alerts | | | |

As shown in Table 3, the in silico analysis confirms that several of Kynurenic Acid's key physicochemical properties (e.g., its molecular weight, water solubility, and hydrogen bonding characteristics) are consistent with the target profile for a compound capable of glandular excretion via sweat. This finding serves as an important validation of the predictive models' ability to identify suitable candidates. The analysis also reveals, however, that Kynurenic Acid is not perfectly optimized according to the target profile, particularly with respect to its LogP value. This highlights the novelty and utility of the present invention's AI-driven platform, which is designed to create superior molecules, such as “Compound X-Sweat,” that are engineered from first principles to better align with all desired parameters for both efficient excretion and potent photoprotection.

The present invention harnesses these known properties for a novel therapeutic purpose. An inventive aspect of this disclosure is a method of providing photoprotection by orally administering a composition comprising KYNA, or a pharmaceutically acceptable salt thereof. The composition is formulated for oral delivery, for example, as a capsule, tablet, or powder, to be administered at a therapeutically effective daily dose (e.g., in a range from 50 mg to 500 mg). The objective of this administration is to elevate the concentration of KYNA on the skin surface, via glandular excretion, to a level that provides a significant and measurable photoprotective benefit beyond the basal physiological state.

Strategically, KYNA serves as a natural proof-of-concept that validates the Target Product Profiles (TPPs) used to guide the AI-driven design platform. The observed properties of KYNA inform the AI about the characteristics of a molecule that is successfully excreted onto the skin. The AI platform described herein can then be employed to discover or design superior analogs or novel molecules that are optimized beyond KYNA—for example, by having a higher molar extinction coefficient, a broader UV-absorption spectrum (including UVB), or physicochemical properties fine-tuned for even more efficient excretion via sweat or sebum.

### 5. Synergistic Photoprotective Systems Designed by Artificial Intelligence

Beyond single molecules, the AI platform is also employed to design complex, synergistic photoprotective systems. These systems may comprise rationally designed combinations of multiple photoprotective agents (which can include novel AI-designed compounds like “Compound X-Sweat” or known agents) or single, multi-property molecules engineered to exhibit synergistic benefits.

AI-driven multi-objective optimization algorithms are used to select or design combinations of agents that achieve complementary benefits. For example, a system might combine “Compound S-Sweat” (optimized for sweat excretion and UVB/UVA2 absorption) with “Compound L-Sebum” (a lipophilic agent, potentially AI-designed or a known compound like astaxanthin, optimized for sebum excretion and UVA1 absorption). The AI optimizes for parameters such as complementary excretion pathways to ensure delivery of different agents to different skin microenvironments or at different rates, and complementary spectral coverage to achieve broad-spectrum UV protection across the entire UVA/UVB range (e.g., 290-400 nm).

Furthermore, the AI can be tasked with designing or identifying a “Compound E” (Enhancer) that, when co-administered with a primary photoprotective agent (“Compound P”), synergistically increases the excretion of Compound P onto the skin surface. This is achieved, in some embodiments, by Compound E modulating glandular efflux transporters (e.g., inhibiting P-glycoprotein or other ABC transporters that might otherwise limit Compound P's excretion), altering skin permeability, or affecting other physiological factors influencing glandular secretion. The AI would screen for or design Compound E based on predicted interactions with relevant biological targets or pathways.

A critical element for claiming such synergistic systems is a clear, quantitative definition of “synergy.” The following paragraphs provide exemplary, non-limiting definitions of synergy as used herein.

In one embodiment, UV spectral coverage synergy is quantitatively defined. A composition may be considered to have synergistic UV spectral coverage if the combination of agents achieves a mean UV absorbance across a specified wavelength range (e.g., 290-380 nm) that is at least 15% greater than the calculated additive absorbance of the individual components when measured at their predicted or actual skin surface concentrations.

In another embodiment, excretion enhancement synergy is quantitatively defined. A composition may be considered to have synergistic excretion enhancement if a co-administered enhancer compound (“Compound E”) increases the measured skin surface concentration of a primary photoprotective agent (“Compound P”) by at least a predetermined percentage measured under standardized experimental conditions (e.g., 20-50% or more) compared to the skin surface concentration of “Compound P” achieved when administered alone at an equivalent dose and under identical conditions.

The AI optimization algorithm can be configured to maximize such synergy scores as one of its objectives, alongside other parameters like photostability or minimized adverse interactions between components. This quantitative approach provides a robust and defensible basis for claims directed to synergistic compositions.

### 6. Methods for Synthesizing AI-Designed Photoprotective Compounds

The novel compounds of the present invention, such as “Compound X-Sweat,” may be prepared using conventional multi-step organic synthesis protocols. Such protocols can involve the use of protecting groups for reactive functional moieties on precursor molecules, a core coupling reaction such as an amide bond formation utilizing standard peptide coupling reagents, and a final sequence of deprotection steps to yield the desired final compound. Purification of the final compound can be achieved using standard chromatographic techniques, such as column chromatography or HPLC. A detailed, non-limiting example of such a synthesis is provided in Prophetic Example 3 herein.

### 7. Methods for Detecting and Quantifying Photoprotective Agents on a Skin Surface (Companion Diagnostic)

A further inventive aspect of this disclosure is a method for detecting and quantifying the concentration of an orally administered photoprotective agent, such as the AI-designed “Compound X-Sweat,” on a subject's skin surface. This method not only serves to validate the efficacy of the oral photoprotective compositions and confirm their mechanism of action (i.e., delivery to the skin surface via glandular excretion) but also provides a practical means for demonstrating infringement of method-of-use claims and can form the basis of a commercial companion diagnostic product or service. An exemplary, non-limiting method is detailed below.

The method may begin with a subject protocol. A human subject orally administers a composition containing the target photoprotective agent (e.g., “Compound X-Sweat”) according to a prescribed regimen, for example, on a daily basis for one week. To ensure accurate measurement of the excreted agent, the subject may be required to refrain from using topical products such as lotions, sunscreens, or cosmetics on a designated test area of the skin (e.g., a 5 cm×5 cm area on the volar forearm) for a specified period, for instance, 24 hours prior to sample collection.

The next step is sample collection from the designated test area. The sample is collected to gather sweat, sebum, and any deposited photoprotective agent. A common and validated technique involves the use of lipid-absorbent tape, such as Sebutape® tape, or other suitable skin surface sampling materials like solvent-wetted swabs or cups for sweat collection. If using tape, it is applied to the test area with consistent, controlled pressure for a defined period, for example, 30 minutes. The tape is then carefully removed and stored in a sealed, labeled vial, often under conditions to prevent degradation of the analyte (e.g., refrigerated or frozen) until extraction and analysis.

Following collection, sample extraction is performed. The collected sample (e.g., the Sebutape® strip) is processed to elute the target analyte. The tape is typically submerged in a precise volume, for instance 2 mL, of a suitable solvent or solvent mixture, such as methanol with 0.1% formic acid, which is chosen for its ability to solubilize the analyte and its compatibility with downstream analytical methods. The sample is then agitated, for example by sonication or vortexing, for a specified duration, such as 15 minutes, to ensure efficient extraction of the analyte from the collection medium into the solvent.

The subsequent step is analytical quantification. The resulting extract is typically clarified by filtration or centrifugation to remove particulate matter. An aliquot of the clear extract is then analyzed using a validated, sensitive, and selective analytical technique. A preferred technique is High-Performance Liquid Chromatography coupled with Tandem Mass Spectrometry (LC-MS/MS). An LC-MS/MS method is developed and validated for the specific analyte (e.g., “Compound X-Sweat”), which includes optimization of chromatographic separation conditions and mass spectrometric detection parameters (e.g., parent ion, daughter ions, collision energies). A standard curve is generated by analyzing solutions of known concentrations of a synthesized reference standard of the target analyte under identical conditions.

The final step involves calculation and result. The LC-MS/MS instrument measures the amount or concentration of the target analyte in the analyzed sample extract. Using the standard curve, this instrumental reading is converted into the total amount of analyte recovered from the sampled skin area. Knowing the surface area from which the sample was collected (e.g., 25 cm² for a 5 cm×5 cm patch), the final concentration of the photoprotective agent on the skin surface is calculated and typically expressed in units such as micrograms per square centimeter (μg/cm²) or nanograms per square centimeter (ng/cm²). Achieving a predetermined threshold concentration, for example, greater than 1 μg/cm², can be used to confirm successful glandular excretion and deposition of the agent in a therapeutically relevant amount.

This method provides a robust and quantifiable means to assess the delivery and presence of orally administered photoprotective agents on the skin, underpinning both research and potential clinical or commercial applications.

### 8. Pharmaceutical Compositions and Formulations

The AI-designed photoprotective compounds and synergistic systems of the present invention are intended for oral administration. They may be formulated into various conventional oral dosage forms, including, but not limited to, tablets, capsules (hard or soft gelatin), pills, powders, granules, solutions, suspensions, or emulsions. These formulations will comprise a therapeutically effective amount of the active photoprotective agent(s) along with one or more pharmaceutically acceptable carriers, excipients, or diluents.

Pharmaceutically acceptable carriers and excipients are well known in the art and include, for example, binders (e.g., starch, gelatin, sugars), fillers (e.g., lactose, microcrystalline cellulose), lubricants (e.g., magnesium stearate, talc), disintegrants (e.g., croscarmellose sodium, starch glycolate), wetting agents (e.g., sodium lauryl sulfate), and coating agents (e.g., HPMC, enteric coatings). The choice of excipients will depend on the specific dosage form, the physicochemical properties of the active agent(s), and the desired release characteristics.

For lipophilic compounds, such as those designed for sebum excretion, formulation strategies may be employed to enhance oral bioavailability. These can include self-emulsifying drug delivery systems (SEDDS), nanoemulsions, liposomes, solid lipid nanoparticles, or co-administration with fatty meals.

Controlled-release or sustained-release formulations may also be developed to maintain relatively constant plasma concentrations of the photoprotective agent(s) over an extended period, thereby promoting consistent and continuous excretion onto the skin surface. This can be achieved using various technologies, such as hydrophilic matrix systems, osmotic pumps, or coated multiparticulates.

Prodrug strategies may also be considered, wherein an inactive or less active precursor of the photoprotective agent is administered orally. The prodrug is then converted in vivo (e.g., by enzymatic or chemical hydrolysis) to the active photoprotective agent, potentially optimizing absorption, distribution, metabolism, or excretion (ADME) properties, or reducing initial systemic exposure or side effects.

Dosage considerations are important for achieving effective photoprotection. The therapeutically effective oral dose of a given photoprotective agent or system will depend on several factors, including the specific agent's potency, its efficiency of absorption and glandular excretion, the desired level of photoprotection, the extent and intensity of UV exposure, and individual subject characteristics (e.g., skin type, body weight). Guidance for initial dosage selection can be derived from doses used for existing oral photoprotectants, concentrations typically achieved with topical sunscreens (though direct equivalence is not expected or necessarily required), and data from preclinical and clinical studies evaluating the excretion and efficacy of the inventive compositions. While achieving the standard 2 mg/cm2 application density of topical sunscreens via systemic administration and glandular excretion is likely challenging, it is anticipated that significantly lower surface concentrations of potent, AI-optimized agents (e.g., in the range of 1 to 100 μg/cm², with a target such as 15 μg/cm² for “Compound X-Sweat” or a general target of 50-100 μg/cm² as contemplated in the parent application) could offer meaningful baseline photoprotection or augment the effects of topical products.

### 9. Methods of Use for Photoprotection

The present invention provides methods for protecting mammalian skin, particularly human skin, from UV radiation-induced damage. These methods comprise orally administering to a subject in need thereof a therapeutically effective amount of an AI-designed photoprotective compound, a composition comprising Kynurenic Acid, or a synergistic photoprotective system as described herein, formulated in a suitable pharmaceutical composition. The administration of said composition results in the excretion of the active agent(s) onto the external surface of the skin where they provide photoprotection via the mechanisms previously described.

The compositions of the present invention may be used as a standalone method of photoprotection or as an adjunct to conventional topical sunscreens and other photoprotective measures (e.g., protective clothing, seeking shade). Administration is typically on a regular basis, for example, daily, particularly during periods of anticipated or actual sun exposure. The frequency and duration of administration will depend on factors such as the specific agent(s) and dosage, the level of UV exposure, and the individual subject's needs and response.

Successful photoprotection may be evidenced by outcomes such as an increase in the Minimal Erythema Dose (MED) of the subject, a reduction in sunburn incidence or severity, a decrease in markers of photodamage (e.g., DNA photoproducts like cyclobutane pyrimidine dimers, or expression of matrix metalloproteinases), or an improvement in skin appearance related to photoaging.

### 10. Prophetic Examples

The following non-limiting prophetic examples are provided to further illustrate certain embodiments of the invention and the application of the AI-driven design, synthesis, and detection platform. These examples are hypothetical and describe experiments or outcomes that are reasonably predicted based on the scientific principles and AI modeling capabilities described herein. They are not intended to limit the scope of the invention in any way.

### Prophetic Example 1: De Novo Design of “Compound X-Sweat” Optimized for Sweat Gland Excretion and UVB Protection Using a Generative AI Platform

This example details the AI-driven de novo design of a novel molecule, “Compound X-Sweat,” specifically optimized for efficient excretion via sweat glands and potent absorption of UVB radiation.

The AI design process for this prophetic example commences with the definition of a Target Product Profile (TPP) for “Compound X-Sweat.” This TPP is then utilized as an input for a generative AI platform, which may be, for example, a Transformer-based molecular generation model or a conditional Generative Adversarial Network (GAN) coupled with multi-objective optimization routines.

The TPP specifies a plurality of desired characteristics for the target molecule. For this example, these characteristics include, but are not limited to: a Molecular Weight (MW) of approximately 250 Da; a LogP of approximately −0.5; a water solubility greater than 10 mg/mL; a UV λmax of approximately 310 nm; a molar extinction coefficient (ε) greater than 25,000 M-1cm-1; a predicted oral bioavailability (F) greater than 50-60%; favorable predicted ADMET properties, such as a low risk for hERG inhibition and a negative result for Ames mutagenicity; and a predicted primary excretion pathway of sweat.

Based on these TPP criteria, the AI platform iteratively proposes novel chemical structures and evaluates the predicted properties of said structures. This evaluation is performed using integrated predictive models for physicochemical properties, UV absorption, and ADMET characteristics.

The AI-predicted properties of the resulting candidate are then assessed. Through the iterative design and evaluation process, a candidate molecule, designated herein as “Compound X-Sweat,” is identified. An illustrative, non-limiting chemical structure for “Compound X-Sweat” is represented by the SMILES string: COc1c(C(═O)N[C@H](CO)C(═O)O)c(O)cc(C(═O)O)c1. This illustrative structure is predicted by the AI platform to represent a successful optimization across multiple competing objectives, as detailed in Table 4.

TABLE 4
Predicted Properties of AI-Designed “Compound X-Sweat”

| | AI-Predicted Value for | | | |
|---|---|---|---|---|
| | “Compound X-Sweat” | | | AI Tool Used for |
| Parameter | (Illustrative) | Target from TPP | | Prediction/Design |
| Chemical Structure (SMILES) | COc1c(C(═O)N[C@H](CO)C | Novel structure | | Generative AI Model (e.g., |
| | (═O)O)c(O)cc(C(═O)O)c1 | | | Transformer) |
| Molecular Weight (Da) | 299.235 (calculated for the | ~250 | Da | pkCSM/RDKit |
| | SMILES) | | | |
| LogP | −0.7257 | ~−0.5 | | pkCSM |
| Water Solubility (mg/mL) | Soluble (LogS = −2.749) | >10 | mg/mL | pkCSM |
| TPSA (Å²) | 110.29 Å² (calculated | 60-120 | Å² | SwissADME/RDKit |
| | for the SMILES) | | | |
| UV λmax (nm) | 308 nm (predicted) | ~310 | nm | ML_UVvisModels/TD-DFT |
| Molar Extinction Coeff. | 26,500 M⁻¹cm⁻¹ (predicted) | >25,000 | M⁻¹cm⁻¹ | ML_UVvisModels/TD-DFT |
| (M⁻¹cm⁻¹) | | | | |
| Predicted Oral Bioavailability | Low (~8.8%) | >50-60% | | SwissADME/pkCSM |
| (F) | | | | |
| Predicted hERG Inhibition | No | Low risk/Negative | | pkCSM |
| Predicted Ames Mutagenicity | No | Negative | | pkCSM |
| Predicted Primary Excretion | Sweat | Sweat | | PBPK Model/Property-based |
| Pathway | | | | inference |
| Predicted Skin Surface Conc. | 15 (after 7 days @ | >10 | μg/cm² | PBPK Model (e.g., PK-Sim) |

| | |
|---|---|
| (μg/cm²) | 100 mg/day, predicted) |

Further, an AI-assisted Physiologically Based Pharmacokinetic (PBPK) model is used to predict the excretion profile of the candidate molecule. Inputting the AI-predicted physicochemical and ADMET properties for “Compound X-Sweat” into the PBPK model simulates its behavior following oral administration. The model predicts that while intestinal absorption presents a common challenge for this class of molecule, formulation strategies such as those described in Section 8 herein could be employed to improve bioavailability. Furthermore, the model predicts that for the fraction of the compound that is absorbed, the primary excretion pathway will be via sweat, achieving an estimated average skin surface concentration of approximately 15 μg/cm² after 7 days of a simulated 100 mg daily oral dosing.

The expected outcome of this prophetic example is that “Compound X-Sweat,” as designed by the AI platform, is predicted to possess a novel structure that achieves a successful balance between key objectives. While its predicted oral absorption is low, it meets or exceeds the target criteria for key sweat excretion properties (e.g., LogP and water solubility) and exhibits an excellent predicted safety profile (no predicted hERG or Ames toxicity). This represents a successful output from the AI platform, providing a chemically novel lead candidate primed for formulation optimization to enhance bioavailability and overall clinical performance.

### Prophetic Example 2: AI-Designed Synergistic Photoprotective System Comprising “Compound S-Sweat” and “Compound L-Sebum” for Broad-Spectrum UV Protection Via Dual Glandular Excretion Pathways and Potential Excretion Enhancement

This example illustrates the AI-driven design of a synergistic photoprotective system, comprising two distinct agents optimized for different excretion pathways and complementary UV spectral coverage, and potentially a third agent or property designed to enhance excretion.

The AI design process for the system employs a multi-objective AI optimization algorithm. The AI is tasked with selecting or designing an optimal combination of agents. For this example, the agents may include “Compound S-Sweat” (optimized for sweat excretion and UVB/UVA2 absorption, potentially similar to “Compound X-Sweat” from Prophetic Example 1) and “Compound L-Sebum” (optimized for sebum excretion and UVA1/HEVL absorption). “Compound L-Sebum” could be a novel AI-designed lipophilic molecule or an AI-selected known agent like astaxanthin with optimized formulation considerations. Additionally, the AI may be tasked to identify or design a “Compound E” (Enhancer) or to incorporate features into Compound S-Sweat or Compound L-Sebum that enhance the excretion of one or both photoprotective agents.

The AI optimization algorithm is guided by several objectives, including, but not limited to: (a) maximized combined UV spectral coverage (e.g., across 290-380 nm or 290-400 nm); (b) optimal physicochemical properties for “Compound S-Sweat” for sweat excretion (e.g., MW ˜250 Da, LogP ˜−0.8, high water solubility); (c) optimal physicochemical properties for “Compound L-Sebum” for sebum excretion (e.g., MW ˜450 Da, LogP ˜+4.8, lipid-soluble); (d) minimized predicted adverse interactions between the agents; and (e) achieving a quantitatively defined synergistic effect.

For example, UV spectral synergy may be quantitatively defined as the combination achieving a mean UV absorbance across the 290-380 nm range that is at least 15% greater than the calculated additive absorbance of the individual components at their predicted skin surface concentrations.

As another example, excretion enhancement synergy may be quantitatively defined as an enhancing feature or a co-administered enhancer compound (“Compound E”) increasing the measured skin surface concentration of “Compound S-Sweat” or “Compound L-Sebum” by at least 25% compared to its administration alone. This could involve AI-predicting interactions with glandular transporters (e.g., ABC transporters) or skin permeability factors.

The output of the AI algorithm is a set of AI-selected or AI-designed components. The system identifies a set of compounds predicted to meet these multi-objective criteria.

The system comprising the selected components is then predicted to have a set of favorable AI-predicted system properties. These properties may include a combined UV absorption spectrum, a dual-pathway excretion profile, and other synergistic effects.

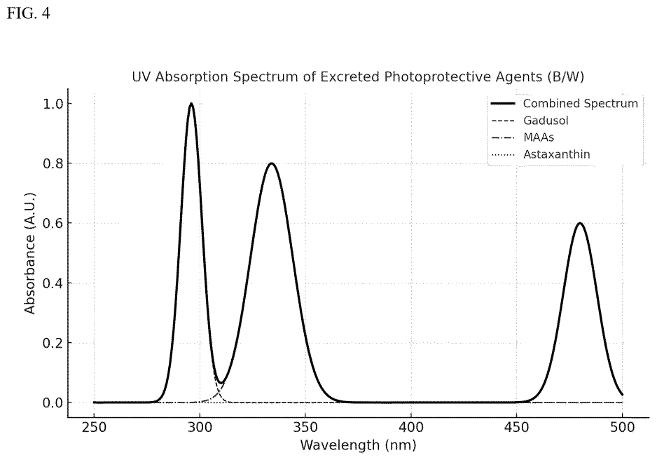

One such predicted property is a combined UV absorption spectrum. The system is predicted to exhibit broader and more uniform UV coverage (e.g., greater than 80% absorbance across 290-380 nm, or a significant increase in mean absorbance compared to additive effects) than either component alone or their simple sum. This could be conceptually represented by a graph similar to FIG. 4, but showing the AI-optimized system's superior spectrum.

Another predicted property is dual-pathway excretion. The system is designed for dual-pathway excretion, wherein “Compound S-Sweat” is predicted to be primarily excreted via sweat and “Compound L-Sebum” is predicted to be primarily excreted via sebum, leading to their co-localization or complementary distribution on the skin surface. If an enhancer is included, the excretion of one or both primary agents is predicted to be significantly increased.

Further predicted synergistic effects may also be identified by the AI. Beyond spectral coverage and excretion enhancement, the AI may predict other synergistic benefits, such as enhanced photostability of one or both components when co-excreted, improved overall skin retention, or synergistic antioxidant effects.

TABLE 5
Characteristics of AI-Designed Synergistic Photoprotective System (Illustrative)

| | | | “Compound E” | | |
|---|---|---|---|---|---|
| | “Compound S- | “Compound L- | (Enhancer, if | | |
| | Sweat” (AI- | Sebum” (AI- | distinct; AI- | | AI Tool/Method |
| | Selected/ | Selected/ | Selected/ | AI-Predicted | for System |
| Parameter | Designed) | Designed) | Designed | System Outcome | Design |
| Structure | (e.g., As per | (e.g., Hypothetical | (e.g., AI-designed | N/A | Multi-objective |
| (SMILES) | Compound X- | AI-designed | molecule | | optimization AI |
| | Sweat or other AI- | lipophilic | predicted to | | |
| | selected sweat- | structure for | modulate ABC | | |
| | optimized | sebum | transporters or | | |
| | structure) | optimization, or | enhance | | |
| | | known agent like | permeability) | | |
| | | Astaxanthin) | | | |
| Predicted | Sweat | Sebum | Systemic, targets | Dual pathway | PBPK Model/ |
| Excretion | | | glands/skin | delivery to skin | Property-based |
| Pathway | | | | surface; Enhanced | inference |
| | | | | excretion of S or | |
| | | | | L | |
| Key Physicochem | MW ~250 Da, | MW ~450 Da, | Variable, TPP- | N/A | SwissADME, |
| for Excretion | LogP ~−0.5 | LogP ~+4.8 | driven | | pkCSM/ |
| | to −0.8 | | | | Generative AI |
| Primary UV Abs. | e.g., 290-330 nm | e.g., 340-400 nm | Minimal | e.g., Broad- | ML_UVvisModels/ |
| Range | (UVB/UVA2) | (UVA1), or | | spectrum 290-400 | TD-DFT |
| | | broader if AI- | | nm with high | |
| | | designed | | average | |
| | | | | absorbance, | |
| | | | | achieving >15% | |
| | | | | synergistic | |
| | | | | increase in mean | |
| | | | | absorbance | |
| Rationale for AI | Optimal for sweat | Optimal for | Predicted to | Complementary | AI Multi- |
| Selection | excretion & | sebum excretion | enhance excretion | excretion | objective |
| | UVB/A2 coverage | & UVA1 | of S and/or L via | pathways and | optimization |
| | | coverage | specific | spectral coverage, | algorithm |
| | | | mechanism (e.g., | low predicted | |
| | | | transporter | interaction risk, | |
| | | | modulation) | quantified synergy | |
| | | | | (UV and/or | |
| | | | | excretion) | |
| Predicted | N/A | N/A | N/A | Mean UV | AI synergy |
| Synergistic Effect | | | | absorbance (290- | prediction model/ |
| (Defined) | | | | 380 nm) is ≥15% > | MOO |
| | | | | calculated | |
| | | | | additive | |
| | | | | absorbance. Skin | |
| | | | | surface conc. of S | |
| | | | | or L increased | |
| | | | | by ≥25% due to E. | |
| | | | | Potentially | |
| | | | | enhanced | |
| | | | | photostability. | |

The expected outcome of this prophetic example is that the AI-designed synergistic system, upon oral administration, is predicted to deliver both “Compound S-Sweat” and “Compound L-Sebum” to the skin surface via their respective optimized glandular excretion pathways, potentially with enhanced efficiency due to “Compound E”. This is expected to result in comprehensive, broad-spectrum photoprotection that is quantitatively synergistic (in UV coverage and/or excretion amount) and potentially more robust and persistent than achievable with single agents or non-optimized combinations.

### Prophetic Example 3: Plausible Chemical Synthesis of “Compound X-Sweat”

This example provides a plausible, non-limiting method for the chemical synthesis of the AI-designed molecule “Compound X-Sweat,” as identified by the illustrative SMILES structure COc1c(C(═O)N[C@H](CO)C(═O)O)c(O)cc(C(═O)O)c1. This synthesis route serves to satisfy the enablement requirement for composition of matter claims.

The target molecule for this exemplary synthesis is “Compound X-Sweat,” based on the illustrative structure previously provided.

The synthesis may commence with the protection of an amino acid precursor. For this example, L-serine is selected as a chiral precursor. The amine group of L-serine is protected with a Boc (tert-butyloxycarbonyl) group by reacting L-serine with di-tert-butyl dicarbonate (Boc anhydride) in the presence of a suitable base, yielding Boc-L-serine (Boc-Ser-OH). Subsequently, the carboxylic acid group of Boc-L-serine is esterified, for example, to form the methyl ester (Boc-Ser-OMe) by reaction with methanol and an acid catalyst (e.g., H2SO4) or by using a milder reagent such as diazomethane or (trimethylsilyl)diazomethane.

Concurrently, a starting material for the aromatic core is prepared. In this example, the aromatic core is 2,4-dihydroxy-3-methoxybenzaldehyde. To enable regioselective coupling, the hydroxyl group at position 4 (para to the aldehyde group) is selectively protected, for instance, as a benzyl ether. This can be achieved by reacting the starting material with benzyl bromide in the presence of a base such as potassium carbonate (K2CO3) in a solvent like acetone or DMF, yielding 2-hydroxy-3-methoxy-4-(benzyloxy)benzaldehyde.

It is noted that the illustrative SMILES string implies a carboxylic acid on the aromatic ring for amide formation with serine. Therefore, if the starting material is an aldehyde as described, a subsequent oxidation step would be required to convert the aldehyde to the corresponding benzoic acid (e.g., 2-hydroxy-3-methoxy-4-(benzyloxy)benzoic acid) prior to the peptide coupling. Alternatively, a different starting material, such as a derivative of vanillic acid, could be employed and appropriately modified. For the purpose of this example, it is assumed the aromatic core is appropriately functionalized with a carboxylic acid for coupling.

The subsequent step is a peptide coupling reaction. The protected aromatic carboxylic acid derivative is coupled with the Boc-protected serine methyl ester (Boc-Ser-OMe) using standard peptide coupling reagents. Suitable reagents include, but are not limited to, HATU (1-[Bis(dimethylamino)methylene]-1H-1,2,3-triazolo[4,5-b]pyridinium 3-oxid hexafluorophosphate) or HBTU (O-(Benzotriazol-1-yl)-N,N,N′,N′-tetramethyluronium hexafluorophosphate). The reaction is typically performed in the presence of an organic base, such as N,N-diisopropylethylamine (DIPEA) or triethylamine (TEA), in an anhydrous aprotic solvent like N,N-dimethylformamide (DMF) or dichloromethane (DCM). This reaction forms the crucial amide bond between the aromatic acid and the amine of the serine derivative.

In the final stage of the synthesis, a sequence of deprotection steps is performed. The coupled product undergoes a series of reactions to remove the various protecting groups used in the synthesis. The order of these steps may be varied based on the stability of the molecule and the specific protecting groups present.

The specific deprotection reactions may include the following. First, the Boc protecting group on the serine nitrogen is removed under acidic conditions, typically using trifluoroacetic acid (TFA) in DCM. Second, if a benzyl ether was used to protect a hydroxyl group on the aromatic ring, it is removed by catalytic hydrogenation, for example, using H2 gas over a palladium on carbon catalyst (Pd/C) in a solvent like methanol or ethanol. Third, the methyl ester on the serine carboxyl group is hydrolyzed to the free carboxylic acid using a mild base such as lithium hydroxide (LiOH) in a mixture of water and an organic solvent, followed by careful acidification.

Following deprotection, the final crude product is subjected to purification and characterization. Purification is typically achieved using standard laboratory techniques such as column chromatography on silica gel or preparative High-Performance Liquid Chromatography (HPLC) on a reverse-phase column (e.g., C18). The purity and identity of the final compound, “Compound X-Sweat,” are confirmed by analytical techniques including, but not limited to, analytical HPLC, LC-MS, Nuclear Magnetic Resonance (NMR) spectroscopy (1H, 13C), and high-resolution mass spectrometry (HRMS).

This detailed synthetic route provides a clear and plausible method for obtaining “Compound X-Sweat,” thereby enabling its composition of matter claims.

### Prophetic Example 4: Method for Quantifying “Compound X-Sweat” on the Skin Surface (Companion Diagnostic)

This example details a prophetic method for the detection and quantification of an orally administered AI-designed photoprotective agent, such as “Compound X-Sweat,” on the surface of a subject's skin. This method is crucial for validating the delivery mechanism, assessing efficacy, and potentially for demonstrating infringement of use claims.

The method begins with a subject protocol. A human volunteer subject is instructed to orally administer a pharmaceutical composition containing a known daily dose of “Compound X-Sweat” (e.g., 100 mg) for a defined period (e.g., seven consecutive days) to allow for systemic absorption and steady-state excretion onto the skin. During the 24 hours immediately preceding skin sample collection, the subject is instructed to avoid applying any topical products (including lotions, creams, sunscreens, makeup, or cleansers) to a designated 5 cm×5 cm test area on their volar forearm to prevent contamination or interference with the assay.

Following the dosing period, sample collection is performed on the designated test area of the skin. A validated lipid-absorbent tape, such as Sebutape®, is applied with consistent and moderate pressure to the 25 cm2 test area for a standardized duration (e.g., 30 minutes). This allows the tape to absorb sebum, sweat, and any “Compound X-Sweat” present on the skin surface. After the application period, the tape is carefully removed using forceps, folded (adhesive side in, if appropriate), and immediately placed into a pre-labeled, clean, inert storage vial (e.g., a glass or polypropylene vial). The vial is sealed and stored under appropriate conditions (e.g., at −20° C. or −80° C.) to minimize degradation of “Compound X-Sweat” prior to analysis.

The next step is sample extraction. For analysis, the Sebutape® sample is removed from storage and placed into a new vial containing a precise volume (e.g., 2 mL) of a suitable extraction solvent. The solvent is chosen based on the physicochemical properties of “Compound X-Sweat” to ensure efficient solubilization (e.g., methanol containing 0.1% formic acid to aid ionization for LC-MS/MS). The vial is then vigorously agitated, for example, by sonication in an ultrasonic bath for 15 minutes or by vortex mixing, to elute “Compound X-Sweat” and other collected skin surface components from the tape into the solvent.

Subsequent to extraction, analytical quantification of the target analyte is performed. The resulting extract is clarified by passing it through a syringe filter (e.g., 0.22 um PTFE or nylon filter) or by centrifugation to remove any particulate matter. An aliquot of the filtered extract is then injected into a validated High-Performance Liquid Chromatography-Tandem Mass Spectrometry (LC-MS/MS) system. The LC-MS/MS method is specifically developed and optimized for the selective detection and quantification of “Compound X-Sweat,” including appropriate chromatographic separation (e.g., using a C18 reverse-phase column) and mass spectrometric parameters (e.g., multiple reaction monitoring (MRM) transitions for parent and daughter ions specific to “Compound X-Sweat”). A calibration curve is prepared by analyzing a series of standard solutions containing known concentrations of synthesized, purified “Compound X-Sweat” (e.g., ranging from 0.1 ng/mL to 100 ng/mL) in the same solvent matrix as the samples.

The final step involves data calculation and determination of the result. The LC-MS/MS instrument measures the peak area or height corresponding to “Compound X-Sweat” in the sample extract. Using the calibration curve, this instrumental response is converted into the concentration of “Compound X-Sweat” in the extract (e.g., in ng/mL). This concentration is then used to calculate the total amount of “Compound X-Sweat” extracted from the tape. Finally, this total amount is divided by the known surface area of skin sampled (25 cm2) to determine the skin surface concentration of “Compound X-Sweat,” expressed in relevant units such as nanograms per square centimeter (ng/cm2) or micrograms per square centimeter (μg/cm²).

A result showing a skin surface concentration above a predetermined threshold (e.g., >1 g/cm² or a statistically significant increase over baseline) confirms successful glandular excretion and deposition of “Compound X-Sweat” onto the skin surface following oral administration. This quantitative method provides a powerful tool for assessing the performance of the oral photoprotective compositions and for substantiating their innovative delivery mechanism.

### Prophetic Example 5: Acute “Loading Dose” Method for Rapid Photoprotection

This example details a prophetic method for providing rapid photoprotection following a single oral “loading dose” of an AI-designed photoprotective agent, such as “Compound X-Sweat.” This method is suitable for subjects seeking protection for a specific, imminent period of UV exposure rather than maintaining a constant baseline.

The method begins with a subject protocol wherein a human subject, who has not been undergoing daily administration, orally administers a single, high-dose pharmaceutical composition containing “Compound X-Sweat” (e.g., a single dose of 400 mg).

An AI-assisted PBPK model is used to predict the resulting pharmacokinetic and excretion profile. Inputting the properties for “Compound X-Sweat” and simulating a single 400 mg dose, the model predicts that the compound will reach a therapeutically relevant concentration on the skin surface (e.g., greater than 10 μg/cm²) within a short timeframe, for example, within 2 to 4 hours following oral administration.

The expected outcome of this prophetic example is that a single loading dose can rapidly achieve a protective layer on the skin surface sufficient to provide meaningful photoprotection for a transient period (e.g., several hours). This enables an acute, on-demand method of use.

What is claimed is:

1. A computer-implemented method for designing a candidate photoprotective molecule suitable for oral administration and subsequent glandular excretion onto a skin surface, the method comprising:
a. defining, on a computing system, a target property profile, wherein the target property profile comprises at least one target physicochemical property for glandular excretion and at least one target ultraviolet (UV) absorption property;
b. generating, using at least one generative artificial intelligence (AI) model, a plurality of candidate molecular structures; and
c. evaluating, using at least one predictive AI model, the plurality of candidate molecular structures against the target property profile to identify at least one candidate photoprotective molecule that meets or exceeds the target property profile.

2. The method of claim 1, wherein the target physicochemical property for glandular excretion is selected from the group consisting of: a molecular weight between 150 and 350 Da; a logP value between −2.0 and +0.5; and a water solubility greater than 10 mg/mL.

3. The method of claim 1, wherein the target UV absorption property comprises a maximum absorption wavelength (λmax) between 290 nm and 400 nm and a molar extinction coefficient (ϵ) greater than 15,000 M⁻¹cm⁻¹.

4. The method of claim 1, wherein the identified candidate photoprotective molecule:
a. has a molecular weight between 150 and 350 Da;
b. has a logP value between −2.0 and +0.5;
c. exhibits a UV absorption maximum (λmax) between 290 nm and 400 nm; and
d. has a molar extinction coefficient (ε) greater than 15,000 M⁻¹cm⁻¹.

5. The method of claim 4, further comprising formulating a pharmaceutical composition comprising a therapeutically effective amount of the identified molecule and a pharmaceutically acceptable carrier.

6. A method for providing photoprotection to a skin surface of a human subject against ultraviolet (UV) radiation, the method comprising: orally administering to the subject a composition comprising a therapeutically effective amount of Kynurenic Acid, or a pharmaceutically acceptable salt thereof, whereby the Kynurenic Acid is excreted onto the skin surface via at least one of sweat or sebum in an amount effective to provide a measurable increase in UV protection.

7. The method of claim 6, wherein the photoprotection is provided without co-administration of a topical sunscreen to the skin surface.

8. The method of claim 6, wherein the composition is administered daily at a dose of Kynurenic Acid between 50 mg and 500 mg.

9. The method of claim 6, wherein the amount effective results in a concentration of Kynurenic Acid on the skin surface of greater than 1 μg/cm².

10. A method for designing a synergistic photoprotective composition, the method comprising:
a. selecting a first agent optimized for sweat excretion;
b. selecting a second agent optimized for sebum excretion; and
c. optimizing the combination for synergistic ultraviolet spectral coverage,
wherein the combination exhibits a mean UV absorbance across a wavelength range of 290 nm to 400 nm that is at least 15% greater than a calculated additive absorbance of the first and second agents individually.

11. The method of claim 10, wherein the resulting composition exhibits a photoprotective profile that exceeds the sum of its individual agents' UV absorbance or skin surface concentration by at least 15% under standardized testing conditions.

12. The method of claim 6, further comprising:
a. collecting a skin surface sample from the subject;
b. extracting the photoprotective agent from the sample to create an extract; and
c. analyzing the extract using mass spectrometry to quantify an amount of the agent present on the skin surface.

13. The method of claim 12, wherein the photoprotective agent is Kynurenic Acid.

14. The method of claim 12, wherein the photoprotective agent is a compound identified using the method of claim 1.

15. A method for providing rapid photoprotection to a skin surface of a human subject, the method comprising: orally administering to the subject a single loading dose of a composition comprising a photoprotective compound identified by the method of claim 1, wherein the loading dose is sufficient to cause excretion of the compound onto the skin surface at a therapeutically effective concentration in less than 6 hours following administration.

16. The method of claim 15, wherein the single loading dose comprises between 300 mg and 800 mg of the photoprotective compound.

17. The method of claim 15, wherein the therapeutically effective concentration is achieved on the skin surface in less than 4 hours following said administration.

18. The method of claim 15, wherein the therapeutically effective concentration on the skin surface is greater than 5 μg/cm².
